(19) United States
(12) Patent Application Publication
(10) Pub. No.: US 2026/0092250 A1
(43) Pub. Date: Apr. 2, 2026

(54) Using The Concepts of Metabolic Flux Rate Calculations and Limited Data to Direct Cell Culture Media Optimization and Enable the Creation of Digital Twin Software Platforms

(71) Applicant: Metalytics, Inc., Cary, NC (US)

(72) Inventors: Eric Jerome Cumming, Durham, NC (US); Samuel Yenne, Raleigh, NC (US); Daniel Isaac Benjamin, San Jose, CA (US); Andy Wiranata Wijaya, Ecublens (CH); Thomas J. Diprospero, Durham, NC (US)

(73) Assignee: Metalytics, Inc., Cary, NC (US)

(21) Appl. No.: 19/113,443

(22) PCT Filed: Sep. 22, 2023
(86) PCT No.: PCT/US2023/074897

§ 371 Date: Mar. 20, 2025

Related U.S. Application Data
(60) Provisional application No. 63/376,798, filed on Sep. 23, 2022.

(51) Int. Cl. C12N 5/00; G16B 5/00; G16B 40/20

(52) U.S. Cl. CPC C12N 5/0018; G16B 5/00; G16B 40/20

(57) ABSTRACT

Provided herein are methods and systems for optimizing media and/or feeding regimes for cells grown in culture by utilizing the analysis of spent cell media in combination with Metabolic Flux Analysis (MFA) and compositions derived from using the same.

### RELATED APPLICATION DATA

The present application claims priority from and the benefit of U.S. Provisional Patent Application No. 63/376,798, filed Sep. 23, 2023, the disclosure of which is hereby incorporated herein by reference in its entirety.

### FIELD

The present inventive concept is related to improved methods and systems for utilizing spent cell media analysis and compositions derived from using the same.

### BACKGROUND

Analyzing spent media for amino acids, other raw material inputs (such as vitamins, fatty acids, sugars, etc.), and cellular by-products reveals substances that growing cells have consumed and/or excreted into the media. However, the simple depletion of raw materials or excretion of by-products provides an incomplete picture regarding how these raw material inputs are processed and utilized by the cells. Methods that more accurately assess consumption and utilization of these raw materials are desired to support cell growth and/or production of desired product(s) by these cells.

### SUMMARY

Embodiments of the present inventive concept provide methods of utilizing spent cell media analysis with or without cell extracts analysis for metabolic flux analysis (MFA) to direct cell-specific media optimization, control of fermentation systems or any other cell culture process and/or direct a cell-specific feeding regime for cultured cells including using computer-assisted methods to calculate metabolic flux rates.

Embodiments of the present inventive concept also provide cell culture media, cell culture media components, and cell culture medium supplements, produced or developed by the methods described herein.

Embodiments of the present inventive concept also provide cell-specific feeding regimes developed by using the methods described herein.

Embodiments of the present inventive concept further provide systems including a computer program and computer-implemented methods to calculate metabolic flux rates.

Embodiments of the present inventive concept further provide cell strain or cell clone selection.

Embodiments of the present inventive concept further provide predictions for genetic alterations.

Embodiments of the present inventive concept further provide predictions for diagnosing diseases and informing drug treatment strategies.

Embodiments of the present inventive concept provide a method for accurately predicting metabolic fluxes using only 4 time-course media measurements and VCD. This is made possible via a 13C MFA-constrained solution.

Accordingly in an aspect of the inventive concept, provided is a method for predicting flux rate of a metabolite of interest in a cell comprising: obtaining quantitative measurements or analytical values for components in spent media or cell extracts; inputting an indication of the quantitative measurements or the analytical values into a trained neural network, wherein the trained neural network characterizes metabolic flux in the cell based on the quantitative measurements or the analytical values; and obtaining a predicted flux rate for the metabolite of interest as an output from the trained neural network, wherein the output is utilized to characterize how the cell behaves or grows under a first condition.

In another aspect of the inventive concept, provided is a system for predicting flux of a metabolite of interest in a cell comprising: at least one processor; a circuit coupled to the at least one processor and configured to input quantitative measurements or analytical values for components in spent media or cell extracts; a circuit configured to characterize the quantitative measurements or analytical values for components in spent media or cell extracts with a trained neural network; an input/output (I/O) circuit coupled to the at least one processor; a storage circuit coupled to the at least one processor and configured to store data and parameters; and a memory coupled to the at least one processor comprising computer-readable program code stored in the memory that when executed by the at least one processor causes the at least one processor to perform operations comprising:

characterizing quantitative measurements or analytical values for components in spent media or cell extracts; obtaining a predicted flux rate for a metabolite of interest from characterization of the quantitative measurements or analytical values for components in spent media or cell extracts with the trained neural network; and controlling output of a determination of a predicted flux rate for the metabolite of interest by way of the I/O circuit, wherein the output is utilized to characterize how the cell behaves or grows under a first condition.

In another aspect of the inventive concept, provided is a computer-implemented method for predicting flux of a metabolite of interest in a cell comprising: inputting quantitative measurements or analytical values for components in spent media or cell extracts into a trained neural network; retrieving representative data related to cell metabolism, cellular expansion, cellular proliferation, and/or cellular growth metrics from a storage circuit into the trained neural network; characterizing the quantitative measurements or analytical values for components in spent media or cell extracts with the trained neural network, wherein the trained neural network characterizes metabolic flux in the cell based on the quantitative measurements or the analytical values and the representative data related to cell metabolism, cellular expansion, cellular proliferation, and/or cellular growth metrics; outputting a predicted flux rate for a metabolite of interest from characterization of the quantitative measurements or analytical values for components in spent media or cell extracts, wherein the output is utilized to characterize how the cell behaves or grows under a first condition.

### BRIEF DESCRIPTION OF THE DRAWINGS

FIG. 1. Simplified flux map representing central carbon metabolism. Fluxes entering glycolysis, pentose phosphate pathway (PPP), and citric acid cycle (CAC) along with fatty acid and amino acid catabolism.

FIG. 2. Flux map of lower glycolysis and the TCA cycle. The flux rates illustrated in this flux map represent the rate of each reaction in nmoles/unit growth/time and are calculated using proprietary metabolic flux software algorithms.

FIG. 3. Flux estimation procedure with or without use of stable isotope tracers. The process is initialized by selecting a set of “free” fluxes that span the nullspace of the stoichiometric matrix. Starting guesses are provided for all free fluxes to seed the procedure. This enables calculation of all linearly dependent fluxes through stoichiometric mass balancing. In one form of metabolic flux calculations, the flux values may then be substituted into the isotopomer balances derived from stable isotopes to simulate measurable isotopomer abundances. The sum-of-squared residuals (SSR) is calculated, which represents the total deviation between all measured quantities and their model-predicted values. An optimization algorithm is then applied to iteratively adjust the values of the free fluxes until the SSR is minimized. At this point, the flux solution is recovered and various statistical tests can be applied to assess goodness-of-fit and to estimate the uncertainties of the regressed parameters. 13C Flux Analysis in Biotechnology and Medicine (In review) Yi Ern Cheah, Clinton M. Hasenour, Jamey D. Young.

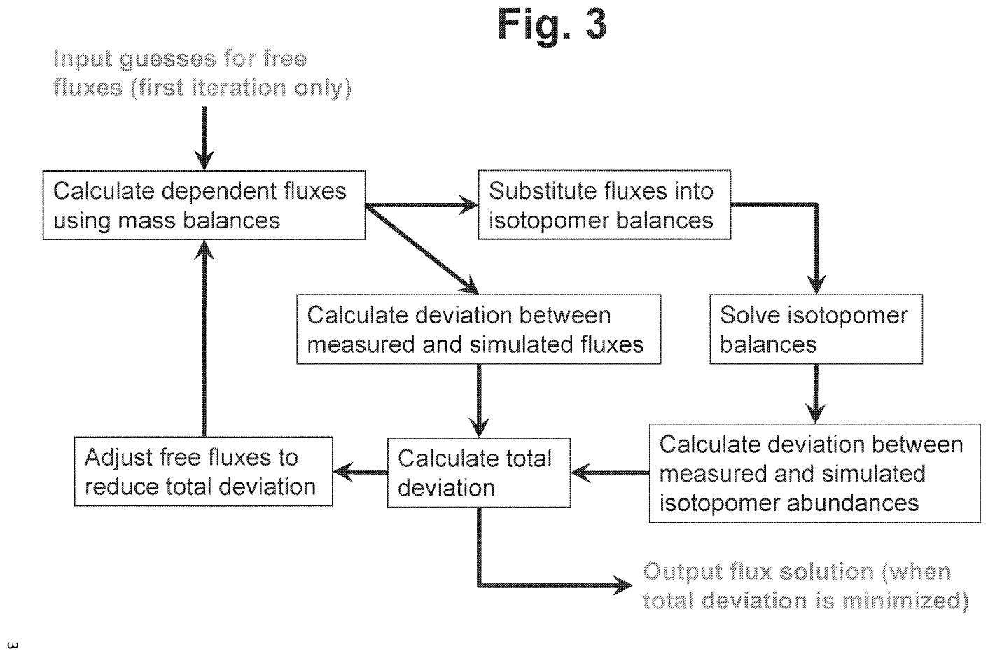

FIG. 4. Metabolic flux analysis reveals lack of available arginine in the early exponential phase of cell line 1. Arginine uptake is occurring in cell lines 1 and 2; however, metabolic flux analysis reveals no catabolism of arginine in the early exponential growth phase of cell line 1. Thus, measurement of the depletion of arginine in the media is insufficient to reveal this metabolic bottleneck.

FIG. 5. Box map of metabolic flux analysis (MFA) according to embodiments of the inventive concept. Predicted flux rates for a metabolite of interest are determined using a machine learning (ML) model.

FIG. 6. Metabolic flux analysis system. Illustrated is a metabolic flux analysis system according to embodiments of the inventive concept.

FIG. 7. Training of a machine learning (ML) model. Illustrated is a machine learning model for metabolic flux analysis according to embodiments of the inventive concept.

FIG. 8. Application of a trained ML model. Illustrated is an example of applying a trained machine learning model to a new observation for predicting metabolic flux rates according to embodiments of the inventive concept.

FIG. 9. Flow chart for application of a trained ML model. Illustrated is a flow diagram of a routine implemented by the metabolic flux analysis system according to embodiments of the inventive concept.

### DETAILED DESCRIPTION

All publications, patents, and patent applications mentioned in this specification are herein incorporated by reference to the same extent as if each individual publication, patent, or patent application was specifically and individually indicated to be incorporated by reference.

In general, described herein are methods and systems for utilizing spent cell media analysis with or without cell extracts analysis for metabolic flux analysis to define and/or optimize culture media supplements and complete culture media compositions derived from using the methods and systems described herein. Methods and systems used herein can also be used to aid in selecting cell strains or clones with desired trait(s), including higher productivity of desired product or reduced production of wasteful byproducts and to aid in identifying genetic alterations for modifying cell strains with the intent of enhancing desired trait(s). Additionally, the methods and systems used herein can also be employed to diagnose diseases and inform drug treatment strategies.

According to embodiments of the present inventive concept, methods include automating a collection of spent media (and cell extract data) and cell growth data that is subjected to computer-implemented analysis to calculate metabolic flux rates. Specifically, the uploaded analytical data is subjected to a proprietary set of algorithms and regressions to calculate a limited number of metabolic flux rates, with or without the use of stable isotope tracers. Using the combination of data set generation and computer-assisted processing, with decision tools (machine learning, artificial intelligence, etc.) and/or technical and intelligent use of the output allows for the identification of various media component limitations and/or possible excesses. This information can be used to direct optimization of culture medium for the specific cells as well as provide a feeding regime (i.e., addition of media components) for the specific cells of interest. This information can be used to aid in selecting cell strains or cell clones with improved metabolic characteristics and/or to aid in directing additional genetic modification(s) to further improve metabolic characteristics. This information can also be used to diagnose diseases and inform drug treatment strategies.

“Culture media” and “cell culture media” may refer to solutions used for growing, storing, handling and/or maintaining cells and/or cell lines. Such solutions generally include various factors necessary for cell attachment, growth and maintenance of the cellular environment. For example, a typical solution may include a basal media formulation, various supplements depending on the cell type and, optionally, antibiotics. The culture media may also include at least one of the following components: 1) an energy source; 2) amino acids; 3) vitamins; 4) free fatty acids; 5) trace elements, where trace elements may be inorganic compounds or naturally occurring elements that are typically required at very low concentrations, usually in the micromolar range; 6) hormones and other growth factors; 7) salts and buffers; 8) nucleosides and bases; 9) proteins and tissue hydrolysates; and 10) serum. As used herein, “spent media” refers to culture media or cell culture media in which cells have grown for some period of time.

“Culture media supplement” refers to additives for addition to culture media that enrich the culture media to support cell growth. In some embodiments, the culture media supplement may be an organic compound. Exemplary additives include, but are not limited to, amino acids, vitamins, minerals, organic acids, lipids, carbohydrates, hormones, growth factors, cytokines, attachment factors, antibiotics, extracts (hydrolysates) of other living systems (yeast, plants, etc.) and serum.

In some embodiments, cell cultures as described herein may be grown in systems including an ability to control fermentation and/or direct a cell-specific feeding regime for cultured cells as described herein. The cell cultures as described herein may be batch cultures, fed-batch cultures, perfusion cultures, and the like.

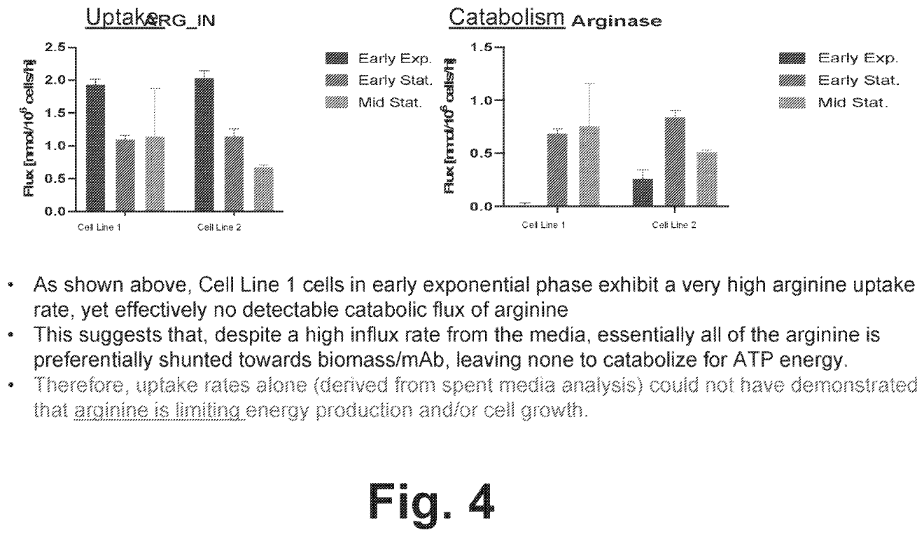

“Metabolic Flux Analysis” or “MFA” refers to the use of experimental measurements and computational modeling in conjunction with complex regression analysis to determine biochemical reaction rates in live biological systems, for example, cells (FIG. 4).

The methods of the present inventive concept may use an MFA methodology including a computer program, e.g., software, software application, script, code, etc., to calculate metabolic flux rates. In some instances, the MFA methodology is exemplified by methodologies developed by Metalytics, Inc. (Cary NC), for example, using a software-enabled technology for metabolic flux analysis for metabolic profiling, or a version thereof, such as, e.g., CoreMFA\hat{ } or MFAtwing\hat{ }. The metabolic profiling may be conducted in combination with analysis of spent media with or without analysis of cell pellet extracts. The results of the analysis of the samples may or may not be automatically uploaded to a computer system for the automated metabolic profiling. In addition, the metabolic profiling may be interpreted with or without the assistance of artificial intelligence or machine learning to facilitate media optimization or the control of fermentation systems or any other means to grow cells in media.

Further, the methods of the present inventive concept can use the results of MFA conducted with stable isotopes to define a solution space of digital twins software to predict metabolic flux rates i.e., MFAtwin™.

A “solution space,” in some embodiments, may include a trained model, selected e.g., with machine learning/artificial intelligence, but is not necessarily limited thereto. The solution space may be constrained, e.g., as a result of training, wherein the constraining is a restriction on the degrees of freedom one has in providing a solution. Constraints are effectively global requirements that restrict the way a system is developed. In some embodiments of the inventive concept, the solution space may be any metabolic flux selected to solve the model, i.e., selected to predict the metabolic flux(es) of interest in the system. When the solution space is constrained (or defined), the degrees of freedom the model has to select a particular metabolic flux rate is limited, meaning the model has to pick from a smaller pool of possible metabolic flux values.

“Defined solution space” refers to the range of possible values that could be used within a metabolic model.

A trained model (with constrained solution space), as described herein, may be obtained using AI or any other algorithmic method that would be appreciated by one of skill in the art.

A “Digital twin” is a digital representation of a physical object, person, or process, contextualized in a digital version of its environment. Digital twins can help an organization simulate real situations and their outcomes, ultimately allowing it to make better decisions. The digital twins of the inventive concept may refer to harmonization and packaging together of a metabolic model, a defined metabolic solution space, and allows for the input of measured experimental data to predict metabolic flux rates within the defined solution space. The output of a digital twin are predictive flux rates constrained by the experimentally measured inputs and solution space.

In some embodiments, the digital twin is used as a tool for side-by-side analysis of a culture system either while a cell culture is active or as a post-culture analysis in which the output of the digital twin is specific to the culture being analyzed. The insights provided from the digital twin system can either be applied to an active culture or future cultures with similar features to that which the digital twin analyzed. The generation of a digital twin requires training and validating data sets that are used to refine the metabolic model's solution space while solving the metabolic model based upon independent input variables. The training and validating data sets are also used to generate flux outputs in which the user or other analytical tool can confirm the successful training of the model with the validation data sets. The predetermined output of a model and solution space will be calculated flux rates, while the output of the digital twin has a wider range for each application. For example, a digital twin output can be reporting the flux rates, interpretation and graphical analysis of the flux rates, or execution of external factors to make corrections to an active culture system.

In some embodiments of the inventive concept, the method and platform/system utilize the concepts of metabolic flux rate calculations and analysis (MFA) within a digital twin to direct cell-specific media optimization, control of fermentation systems or other cell culture systems, a cell-specific feeding regime for cultured cells, predict cell strain or cell clone selection, inform appropriate genetic modifications, maximize bioproduction output, and/or diagnose, inform, and treat diseases and disease states as described herein.

In some embodiments, the system for predicting metabolic flux rates in a culture system may include, e.g., a metabolic model, a trained model/metabolic solution space, a means for inputting measured experimental data into the system, and a means for generating predictive flux rates within the trained model/metabolic solution space based on the input experimental data and the metabolic model. In some embodiments, the predicted flux rates are constrained by the experimentally measured inputs of the trained model/metabolic solution space. In some embodiments, the system may include a means for performing side-by-side analysis of a culture system, either during an active cell culture or as a post-culture analysis and generating culture-specific output. In some embodiments, the culture-specific output includes insights applicable to an active culture system or future cultures that share features with the culture analyzed by the system.

In some instances, a computer can be used to store and process the data. A computer-executable logic can be employed to perform such functions as grouping and/or analyzing the data. A computer can be useful for displaying, storing, retrieving, or calculating diagnostic results from the molecular profiling; displaying, storing, retrieving, or calculating raw data; or displaying, storing, retrieving, or calculating any sample information useful in the methods described herein. Provided herein are systems including computer readable instructions for performing methods described herein. Provided herein are computer readable medium including instructions which, when executed by a computer, cause the computer to perform methods described herein.

“Metabolic models” refer to a mapped connection of enzymatic pathways in which the flow of nutrients can be tracked within the mapped system. Metabolic models can map entire enzymatic chains in which the substrate(s) and product(s) of the enzymatic reaction follow one to another; or metabolic models can be simplified enzymatic chains where assumptions of the substrate-product flow are assumed to follow known reaction paths defined in current literature. Metabolic models also encompass the use of enzyme kinetics and enzymatic properties in which substrate-product concentrations can be used and calculated.

In some embodiments, the metabolic model may include a mapped connection of enzymatic pathways, wherein the flow of nutrients within the mapped system can be tracked from input to output and final products, including “biomass.” In some embodiments, the metabolic model may include at least two (2) nodes, where at least one (1) node is constrained by measured experimental data. In some embodiments, the metabolic model may include enzymatic pathways, wherein the enzymatic pathways map entire enzymatic chains or simplified enzymatic chains following known reaction paths defined in current literature, or even reaction paths heretofore unknown. In some embodiments, the metabolic model may be characterized using enzyme kinetics and/or enzymatic properties to determine substrate-product concentrations.

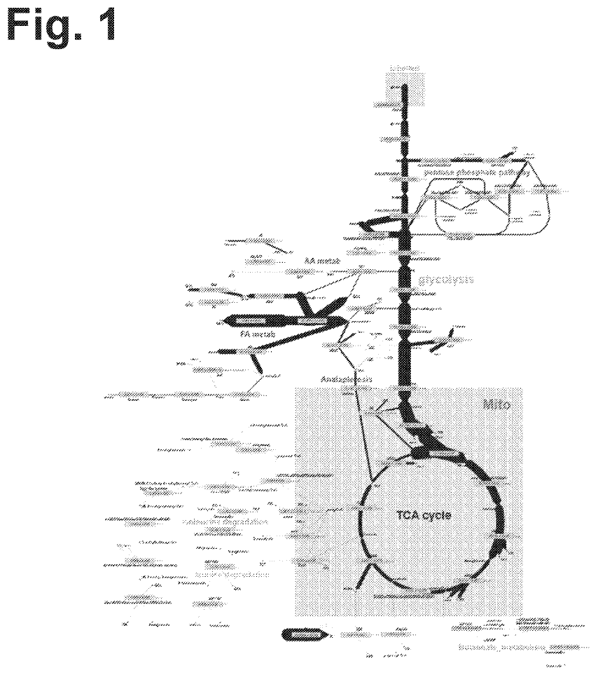

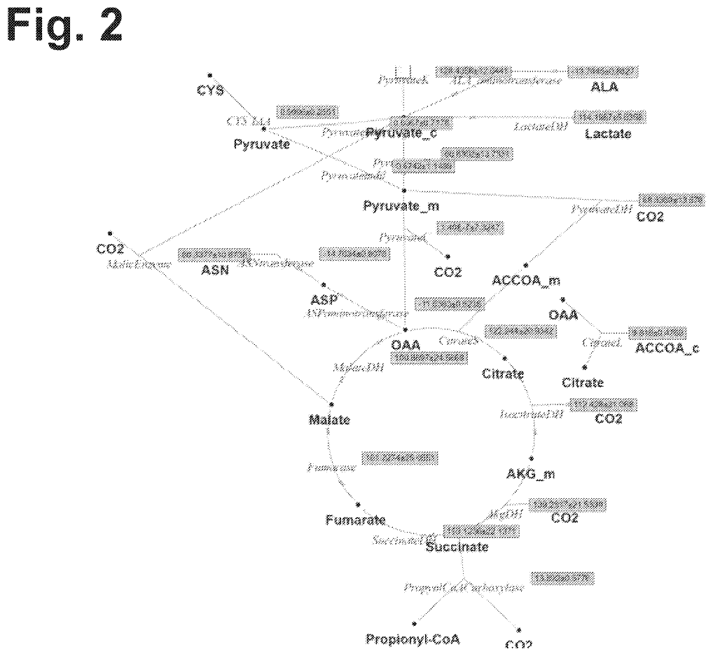

Notably, a metabolic model, e.g., pathways as illustrated in various forms in FIGS. 1 and 2 will be constructed for use to perform MFA. In an exemplary embodiment, the metabolic model is represented by central carbon metabolism through amino acid catabolism. Other embodiments of the metabolic model may represent different metabolic pathways or combinations of pathways. The metabolic model may also include the uptake and/or excretion of amino acids, carbohydrates, and other components used to support cell growth or metabolic activity. All the chemical transitions may be represented as algorithms. Examples of which are shown in Table 1.

In addition, the metabolic model also includes a combined biomass formation reaction that drains anabolic precursors derived from spent media and central carbon metabolism for the biosynthesis of cellular macromolecules. The stoichiometric coefficients for this biomass equation are unique to each cell type of interest and depend on the macromolecular composition of the cell. Using this model, along with the data described above, metabolic flux rates will be calculated with a form of a proprietary metabolic flux software.

TABLE 1
Exemplary chemical reactions are represented as algorithms for metabolic flux calculations. All atom transitions are captured in algorithms.

| Pathway | Reaction | Active.f | Active.b | Reducts | Direction | Products |
|---|---|---|---|---|---|---|
| Glycolysis | Bexokinase | TRUE | NA | Glucose (C1 [symbol] a1_1 C2 [symbol] a2_1 C3 [symbol] a3_1 | −> | G6P (C1 [symbol] a1_1 C2 [symbol] a2_1 C3 [symbol] a3_1 |
| | | | | C4 [symbol] a4_1 C5 [symbol] a5_1 C6 [symbol] a6_1) | | C4 [symbol] a4_1 C5 [symbol] a5_1 C6 [symbol] a6_1) |
| Glycolysis | PyruvateK | TRUE | NA | PEP (C1 [symbol] a1_1 C2 [symbol] a2_1 C3 [symbol] a3_1) | −> | Pyruvate_c (C1 [symbol] a1_1 C2 [symbol] a2_1 |
| | | | | | | C3 [symbol] a3_1 |
| Glycolysis | Phospho- | TRUE | NA | FructoseBP (C1 [symbol] a1_1 C2 [symbol] a2_1 | −> | DHAP (C1 [symbol] a5_1 C2 [symbol] a2_1 C3 [symbol] a6_1) + |
| | fructokinase | | | C3 [symbol] a1_1 C4 [symbol] a4_1 C5 [symbol] a5_1 C6 [symbol] a6_1) | | G3P (C1 [symbol] a4_1 C2 [symbol] a1_1 C3 [symbol] a3_1) |
| Glycolysis | GADPH | TRUE | TRUE | G3P (C1 [symbol] a1_1 C2 [symbol] a2_1 C3 [symbol] a3_1) | <−> | 3PG (C1 [symbol] a2_1 C2 [symbol] a3_1 C3 [symbol] a1_1) |
| Glycolysis | Triose Phosphate | TRUE | TRUE | DHAP (C1 [symbol] a1_1 C2 [symbol] a2_1 C [symbol] a3_1) | <−> | G3P (C1 [symbol] a1_1 C2 [symbol] a2_1 C3 [symbol] a3_1) |
| | Isomerase | | | | | |
| Glycolysis | PhosphoglucoseI | TRUE | TRUE | G6P (C1 [symbol] a1_1 C2 [symbol] a2_1 C3 [symbol] a3_1 | <−> | Fructose6P (C1 [symbol] a1_1 C2 [symbol] a6_1 |
| | | | | C4 [symbol] a4_1 C5 [symbol] a5_1 C6 [symbol] a6_1) | | C3 [symbol] a2_1 C4 [symbol] a3_1 C3 [symbol] a4_1 C6 [symbol] a3_1) |
| Glycolysis | FructoseBP | TRUE | TRUE | FructoseBP (C1 [symbol] a1_1 C2 [symbol] a2_1 | <−> | Fructose6P (C1 [symbol] a1_1 C2 [symbol] a2_1 |
| | | | | C3 [symbol] a3_1 C4 [symbol] a4_1 C5 [symbol] a5_1 C6 [symbol] a6_1) | | C3 [symbol] a3_1 C4 [symbol] a4_1 C5 [symbol] a5_1 C6 [symbol] a6_1) |
| Glycolysis | pgm | TRUE | TRUE | 3PG (C1 [symbol] a1_1 C2 [symbol] a2_1 C3 [symbol] a3_1) | <−> | PEP (C1 [symbol] a1_1 C2 [symbol] a2_1 C3 [symbol] a3_1) |
| Glycolysis | PyruvateDH | TRUE | NA | Pyruvate_m (C1 [symbol] a1_1 C2 [symbol] a2_1 | −> | ACCOA_m (C1 [symbol] a1_1 C2 [symbol] a2_1) + |
| | | | | C3 [symbol] a3_1) | | CO2 (C1 [symbol] a3_1) |
| TCA cycle | CitrateS | TRUE | NA | ACCOA_m (C1 [symbol] a1_1 C2 [symbol] a2_1 + | −> | Citrate (C1 [symbol] a1_1 C2 [symbol] a1_2 C3 [symbol] a2_1 |
| | | | | OAA (C1 [symbol] a1_2 C2 [symbol] a2_2 C3 [symbol] a3_2 | | C4 [symbol] a3_2 C5 [symbol] a4_2 C6 [symbol] a2_2) |
| | | | | C4 [symbol] a4_2) | | |
| TCA cycle | isocitrateDH | TRUE | TRUE | Citrate (C1 [symbol] a1_1 C2 [symbol] a2_1 C3 [symbol] a3_1 | <−> | AKG_m (C1 [symbol] a6_1 C2 [symbol] a1_1 C3 [symbol] a2_1 |
| | | | | C4 [symbol] a4_1 C5 [symbol] a5_1 C6 [symbol] a6_1) | | C4 [symbol] a3_1 C5 [symbol] a4_1) + CO2 (C1 [symbol] a5_1) |
| TCA cycle | AkgDH | TRUE | NA | AKO_m (C1 [symbol] a1_1 C2 [symbol] a2_1 C3 [symbol] a3_1 | −> | Succinate (C1 [symbol] a2_1 C2 [symbol] a1_1 C3 [symbol] a4_1 |
| | | | | C4 [symbol] a4_1 C5 [symbol] a5_1) | | C4 [symbol] a3_1) + CO2 (C1 [symbol] a3_1) |

[symbol]

indicates data missing or illegible when filed

In general, the analyses of the spent media will be quantitative such that a change in concentration can be calculated. Another parameter will be the type of compound and/or the specific compounds such as amino acids, glucose and/or lactate. Other compounds that are known to be toxic to cells if they accumulate may be measured. The collected data related to specific metabolites from spent media and/or cell extracts for calculating flux rates may or may not include the use of a stable isotope tracer. As used herein, “stable isotope tracers” refer to molecules including a naturally occurring non-radioactive form of an atom that can be used to track a metabolic substrate through downstream biochemical reactions. Non-limiting examples include hydrogen, carbon, oxygen and nitrogen isotopes. In particular embodiments of the present inventive concept, the stable isotope tracer includes 13C, 15N, 2H or 18O.

The analyses of spent media as described herein may be utilized to determine rates of metabolic flux. The method of determining metabolic flux rates is not particularly limited, and rates may be acquired from quantitative analytical techniques including mass spectrometry, NMR, Ramen spectroscopy, or bioanalytical methods including ELISA, Northern, Southern, Western Blot, electrochemical, fluorescence, luminescence, or colorimetric analysis, etc.

“Cell growth data” refers to indicia of cell growth, maturation and/or productivity as understood by those skilled in the art and include, but are not limited to, an increase in the number of cells, an increase in size, or an increase in weight. This data may be collected visually, with an on-line device such as a cell counter, and/or could be proxy data such as turbidity. In particular embodiments of the present inventive concept, cells include any cells grown in culture, that is, cells grown in suspension or grown adhered to a variety of surfaces or substrates in vessels such as roller bottles, tissue culture flasks, dishes, multi-well plates and the like. Large scale approaches, such as bioreactors, including cells grown attached to microcarriers in stirred fermenters, encompass cells grown in culture.

“Cells” as used herein include prokaryotic and eukaryotic cells. Further, cells refer to plant and animal cells (both human and non-human) and further include invertebrate, non-mammalian vertebrate and mammalian cells. Cells also include bacteria, fungi (such as yeasts) and molds. All such designations include cell populations and progeny. Thus, the terms “transformants” and “transfectants” include the primary subject cell and cell lines derived therefrom without regard for the number of transfers. Exemplary non-mammalian vertebrate cells include, for example, avian cells, reptilian cells and amphibian cells. Exemplary invertebrate cells include, but are not limited to, insect cells such as, for example, caterpillar (Spodoptera frugiperda) cells, mosquito (Aedes aegypti) cells, fruitfly (Drosophila melanogaster) cells, Schneider cells, and Bombyx mori cells. The cells may be differentiated, partially differentiated or undifferentiated, e.g., stem cells, including embryonic stem cells and pluripotent stem cells. Additionally, tissue samples derived from organs or organ systems may be used according to the invention. Exemplary mammalian cells include, for example, cells derived from human, non-human primate, cat, dog, sheep, goat, cow, horse, pig, rabbit, rodents including mouse, hamster, rat and guinea pig and any derivatives and progenies thereof. Exemplary cell lines include, but are not limited to, Sf9 cells, Chinese hamster ovary cells (CHOs), HEK 293 and HeLa cells.

Cellular components, and components in spent media or cell extracts, that may be evaluated in the assessment of cell growth include: organic compounds; organic acids; amino acids, such as alanine, arginine, asparagine, aspartate, citrulline, cystine/cysteine, glutamate, glutamine, glycine, histidine, hydroxyproline, isoleucine, leucine, lysine, methionine, ornithine, phenylalanine, proline, serine, taurine, threonine, tryptophane, tyrosine and valine; sugars, such as glucose and lactate; trace elements such as chromium (Cr), manganese (Mn), iron (Fe), cobalt (Co), nickel (Ni), copper (Cu), zinc (Zn), selenium (Sc), molybdenum (Mo) and cadmium (Cd); minerals; and vitamins, vitaminoids and precursors such as vitamin B1 (thiamine), vitamin B2 (riboflavin), vitamin B3 (nicotinamide), vitamin B5 (calcium pantothenate), vitamin B6 (pyridoxal, pyridoxine), vitamin B7 (biotin), vitamin B9 (folic acid), aminobenzoic acid, vitamin B12 (cyanocobalamin) and vitaminoids such as choline chloride, a-Lipoic acid and Coenzyme Q10. In some embodiments, the cellular components, and components in spent media or cell extracts, that may be evaluated in the assessment of cell growth may include any culture media supplement, e.g., organic compounds; amino acids; fatty acids; sugars; vitamins; minerals; organic acids; growth factors; lipids; carbohydrates; hormones; cytokines; attachment factors; antibiotics; extracts (hydrolysates) of other living systems (yeast, plants, etc.); and serum, as described herein without limitation.

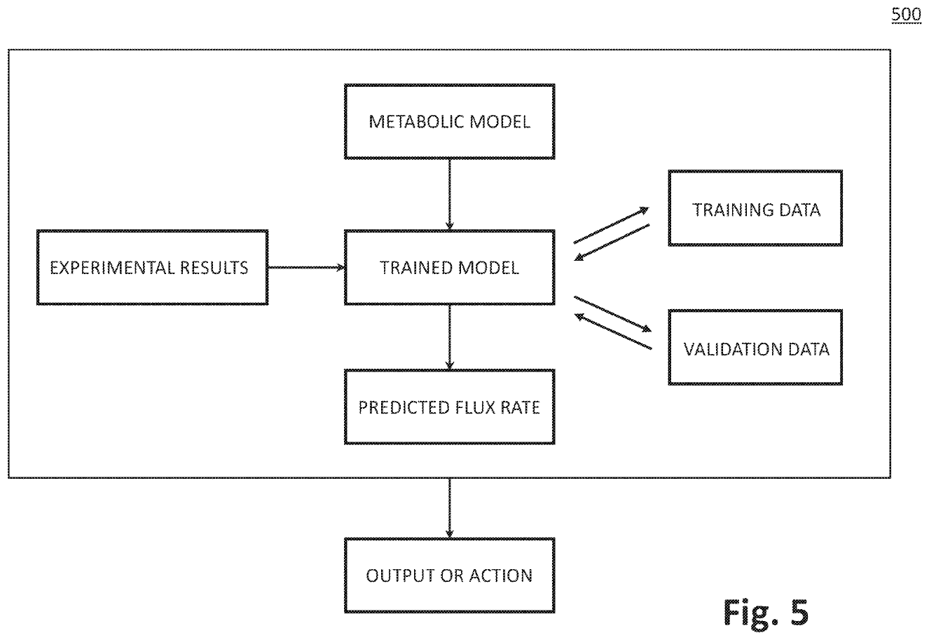

FIG. 5 is a box map of MFA performed in accordance with some embodiments of the inventive concept. A metabolic model, e.g., as described herein, may be selected for training, e.g., with machine learning/artificial intelligence. Training of the model results in a trained model/solution space that is used to analyze experimental results in order to determine predicted flux rates of, for example, a metabolite of interest. This predicted flux rate for the metabolite of interest may then be used to, e.g., facilitate media optimization, control fermentation systems, e.g., provide recommendations to the fermentation system regarding media optimization and/or media supplementing, or any means related to growing cells in media without limitation.

In some embodiments, the model/solution space may include a range of possible metabolic flux rates. Although FIG. 5 is discussed hereinabove in relation to ML/AI training of the model/solution, the solution can arise from any individual point or combinations of the following points, wherein the flux rate model/solution space represents metabolic flux rates of cells within a specific context, e.g., quantified metabolite data derived from intracellular and/or extracellular sources for a specific experimental design, a computationally trained metabolic model, a repository of flux data, inherent enzymatic properties, including but not limited to kinetics, regulation, binding affinity, and/or combinations of genetic mapping with -omics data. In some embodiments, the solution may include supplementing the defined flux rate model/solution with experimental data obtained from a specific cell culture process used to further constrain the model/solution. The number of experimental data points must be greater than or equal to two (2) to further constrain the model/solution. In some embodiments, system and/or model/solution may include a means for training and validating the model using data sets to refine the trained model/solution and generate flux outputs. In some embodiments, training and validating data sets are used to confirm the successful training of the model with validation data sets. In some embodiments, the method, system, and/or model/solution space further includes the use of cellular culture data from collection of spent media, cell extract data, and/or cell growth data. In some embodiments, the predetermined output of the model/solution includes or consists of calculated flux rates. In some embodiments, the output of the methods and/or systems of the inventive concept encompass a range of outputs including reporting flux rates, interpreting and graphically analyzing flux rates, or executing external factors to make corrections to an active culture system. In some embodiments, the methods and/or systems of the inventive concept may utilize both the defined model/solution and experimentally defined measurements to calculate metabolic flux rates. In some embodiments, the method and/or system of the inventive concept further include subjecting spent cell media analysis and/or cell extracts analytical data to a set of algorithms and regressions to calculate a limited number of flux rates, which may be performed with or without the use of a stable isotope tracer. In some embodiments, the methods of the inventive concept may further include the utilization of the calculated metabolic flux rates to optimize cell-specific media composition in a cell culture process. In some embodiments, the methods of the inventive concept may include the utilization of the calculated metabolic flux rates to control fermentation systems in a cell culture process. In some embodiments, the methods of the inventive concept may include the utilization of the calculated metabolic flux rates to direct a cell-specific feeding regime for cultured cells, including the use of computer-assisted methods to calculate metabolic flux rates.

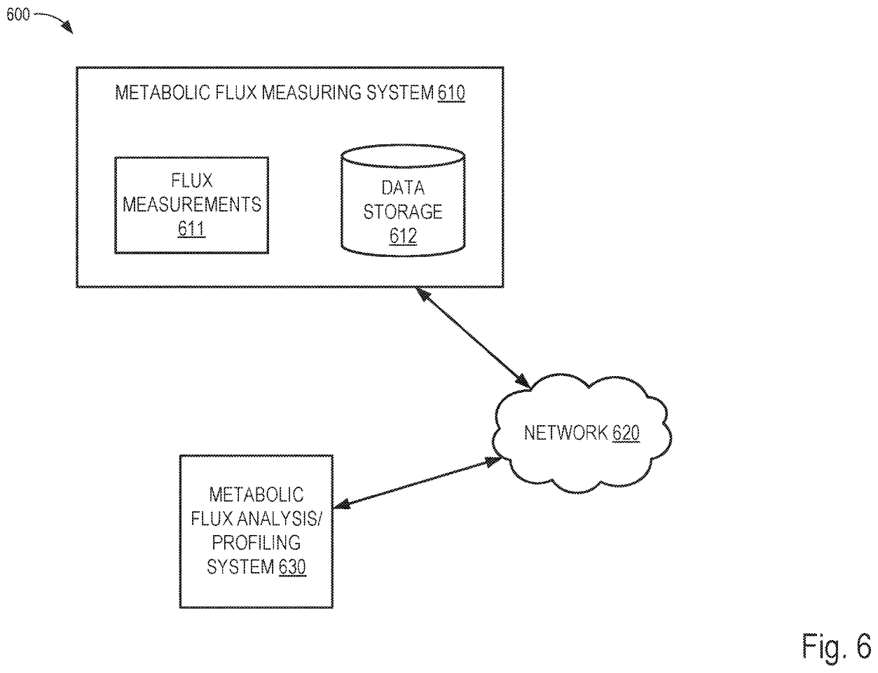

FIG. 6 is a diagram illustrating an exemplary metabolic flux analysis system 600 according to some embodiments of the present inventive concept. The metabolic flux analysis system may include a metabolic flux measuring system 610, which may include a means for measuring and/or obtaining flux measurements 611 for, e.g., cells growing in culture media, and a means for data storage of the measured/obtained metabolic flux values. The metabolic flux values collected with the metabolic flux measuring system 610 may be connected to a metabolic flux analysis/profiling system 630 through a network 620. Alternatively, the metabolic flux measuring system 610 may be connected to/interact with the metabolic flux analysis/profiling system 630 directly.

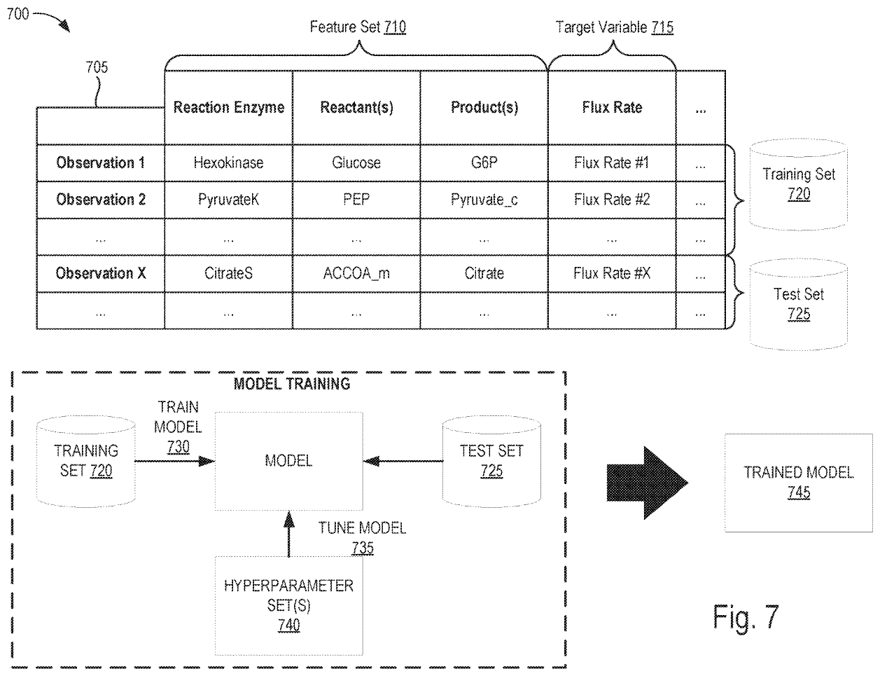

FIG. 7 is a diagram illustrating an example of training a machine learning model 700 in connection with the present inventive concept. The machine learning model training described herein may be performed using a machine learning system. The machine learning system may include or may be included in a computing device, computer program, software, software application, script, code, a server, a cloud computing environment, or the like, and as part of the metabolic flux analysis/profiling system 630 of FIG. 6, may be connected through a network 620 depicted in FIG. 6, to the metabolic flux measuring system 610 shown in FIG. 6. Alternatively, the machine learning system may be connected directly with the metabolic flux measuring system 610 of FIG. 6.

As depicted by reference number 705, a machine learning model may be trained using a set of observations. The set of observations may be obtained and/or input from historical data, such as data gathered during one or more processes described herein. For example, the set of observations may include data gathered from experimental measurements. In some embodiments, the machine learning system may receive the set of observations (e.g., as input) from the experimental measurements or from a storage/computing device. In some embodiments, the set of observations may include data gathered from the metabolic flux measuring system 610 as shown in FIG. 6.

As shown by reference number 810, a feature set may be derived from the set of observations. The feature set may include a set of variables. A variable may be referred to as a feature. A specific observation may include a set of variable values corresponding to the set of variables. A set of variable values may be specific to an observation. In some cases, different observations may be associated with different sets of variable values, sometimes referred to as feature values.

In some embodiments, the machine learning system may determine variables for a set of observations and/or variable values for a specific observation based on input received from the metabolic flux analysis/profiling system 630 shown in FIG. 6. For example, the machine learning system may identify a feature set (e.g., one or more features and/or corresponding feature values) from structured data input to the machine learning system, such as by extracting data from a particular column of a table, extracting data from a particular field of a form and/or a message, and/or extracting data received in a structured data format. Additionally, or alternatively, the machine learning system may receive input from the metabolic flux measurements 611 or from an operator to determine features and/or feature values, which may be stored as data compiled by the metabolic flux measuring system 610 in data storage 612.

As an example, a feature set for a set of observations may include a first feature of a reaction, such as a reaction that is part of a metabolic pathway and is catalyzed by an enzyme, a second feature of a reactant(s) for the reaction/enzyme, a third feature of a product(s) derived from the reaction/enzyme, and so on. As shown, for a first observation, the first feature may be a reaction catalyzed by the enzyme hexokinase, the second feature may be the reactant glucose for hexokinase, the third feature may be the product G6P, which results from hexokinase acting on glucose, and so on. These features and feature values are provided as examples and may differ in other examples. For example, the feature set may include one or more of the following features: a metabolic pathway, a rate for the forward reaction catalyzed by the enzyme, a rate for the reverse reaction catalyzed by the enzyme, a direction/reversibility of the enzyme-catalyzed reaction, and a flux rate (the difference between forward and reverse reactions) for the enzyme-catalyzed reaction, etc. In some embodiments, the machine learning system may pre-process and/or perform dimensionality reduction to reduce the feature set and/or combine features of the feature set to a minimum feature set. A machine learning model may be trained on the minimum feature set, thereby conserving resources of the machine learning system (e.g., processing resources and/or memory resources) used to train the machine learning model. On some embodiments, the machine learning system normalizes values of the feature set prior to analysis of the values.

The set of observations may be associated with a target variable 715. The target variable 715 may represent a variable having a numeric value (e.g., an integer value or a floating point value), may represent a variable having a numeric value that falls within a range of values or has some discrete possible values, may represent a variable that is selectable from one of multiple options (e.g., one of multiples classes, classifications, or labels), or may represent a variable having a Boolean value (e.g., 0 or 2, True or False, Yes or No), among other examples. A target variable may be associated with a target variable value, and a target variable value may be specific to an observation. In some cases, different observations may be associated with different target variable values.

In example 700, the target variable 715 is a flux rate, which has a value of “Flux Rate #1” for the first observation and “Flux Rate #2” for the second observation. Nevertheless, the metabolic flux measurement system may determine flux rates for the same reaction over time. For example, the metabolic flux measurement system may determine flux rates for a reaction catalyzed by a particular enzyme at different time points. The feature set 710 and target variable 715 described above are provided as examples, and other examples may differ from what is described above. It will be understood that the target variable may vary across embodiments. For example, in some embodiments, the target variable 715 may be a production rate of a product, or consumption rate of a reactant.

The target variable may represent a value that a machine learning model is being trained to predict, and the feature set may represent the variables that are input to a trained machine learning model to predict a value for the target variable. The set of observations may include target variable values so that the machine learning model can be trained to recognize patterns in the feature set 710 that lead to a target variable value. A machine learning model that is trained to predict a target variable value may be referred to as a supervised learning model or a predictive model. When the target variable is associated with continuous target variable values (e.g., a range of numbers), the machine learning model may employ a regression technique. When the target variable is associated with categorical target variable values (e.g., classes or labels), the machine learning model may employ a classification technique.

In some embodiments, the machine learning model may be trained on a set of observations that do not include a target variable (or that include a target variable, but the machine learning model is not being executed to predict the target variable). This may be referred to as an unsupervised learning model, an automated data analysis model, or an automated signal extraction model. In this case, the machine learning model may learn patterns from the set of observations without labeling or supervision, and may provide output that indicates such patterns, such as by using clustering and/or association to identify related groups of items within the set of observations.

As further shown, the machine learning system may partition the set of observations into a training data set 720 that includes a first subset of observations of the set of observations, and a validation data set 725 that includes a second subset of observations of the set of observations. The training set 720 may be used to train (e.g., fit or tune) the machine learning model, while the validation data set 725 may be used to evaluate a machine learning model that is trained using the training data set 720. For example, for supervised learning, the validation data set 725 may be used for initial model training using the first subset of observations, and the validation data set 725 may be used to test whether the trained model accurately predicts target variables in the second subset of observations. In some embodiments, the machine learning system may partition the set of observations into the training data set 720 and the validation data set 725 by including a first portion or a first percentage of the set of observations in the training set 720 (e.g., 75%, 80%, or 85%, among other examples) and including a second portion or a second percentage of the set of observations in the validation data set 725 (e.g., 25%, 20%, or 25%, among other examples). In some implementations, the machine learning system may randomly select observations to be included in the training set 720 and/or the validation data set 725.

As shown by reference number 730, the machine learning system may train a machine learning model using the training data set 720. This training may include executing, by the machine learning system, a machine learning algorithm to determine a set of model parameters based on the training set 720. In some implementations, the machine learning algorithm may include a regression algorithm (e.g., linear regression or logistic regression), which may include a regularized regression algorithm (e.g., Lasso regression, Ridge regression, or Elastic-Net regression). Additionally, or alternatively, the machine learning algorithm may include a decision tree algorithm, which may include a tree ensemble algorithm (e.g., generated using bagging and/or boosting), a random forest algorithm, or a boosted trees algorithm. A model parameter may include an attribute of a machine learning model that is learned from data input into the model (e.g., the training set 720). For example, for a regression algorithm, a model parameter may include a regression coefficient (e.g., a weight). For a decision tree algorithm, a model parameter may include a decision tree split location, as an example.

As shown by reference number 735, the machine learning system may use one or more hyperparameter sets 740 to tune the machine learning model. A hyperparameter may include a structural parameter that controls execution of a machine learning algorithm by the machine learning system, such as a constraint applied to the machine learning algorithm. Unlike a model parameter, a hyperparameter is not learned from data input into the model. An example hyperparameter for a regularized regression algorithm includes a strength (e.g., a weight) of a penalty applied to a regression coefficient to mitigate overfitting of the machine learning model to the training data set 720. The penalty may be applied based on a size of a coefficient value (e.g., for Lasso regression, such as to penalize large coefficient values), may be applied based on a squared size of a coefficient value (e.g., for Ridge regression, such as to penalize large squared coefficient values), may be applied based on a ratio of the size and the squared size (e.g., for Elastic-Net regression), and/or may be applied by setting one or more feature values to zero (e.g., for automatic feature selection). Example hyperparameters for a decision tree algorithm include a tree ensemble technique to be applied (e.g., bagging, boosting, a random forest algorithm, and/or a boosted trees algorithm), a number of features to evaluate, a number of observations to use, a maximum depth of each decision tree (e.g., a number of branches permitted for the decision tree), or a number of decision trees to include in a random forest algorithm.

To train a machine learning model, the machine learning system may identify a set of machine learning algorithms to be trained (e.g., based on operator input that identifies the one or more machine learning algorithms and/or based on random selection of a set of machine learning algorithms), and may train the set of machine learning algorithms (e.g., independently for each machine learning algorithm in the set) using the training data set 720. The machine learning system may tune each machine learning algorithm using one or more hyperparameter sets 740 (e.g., based on operator input that identifies hyperparameter sets 740 to be used and/or based on randomly generating hyperparameter values). The machine learning system may train a particular machine learning model using a specific machine learning algorithm and a corresponding hyperparameter set 740. In some implementations, the machine learning system may train multiple machine learning models to generate a set of model parameters for each machine learning model, where each machine learning model corresponds to a different combination of a machine learning algorithm and a hyperparameter set 740 for that machine learning algorithm.

In some embodiments, the machine learning system may perform cross-validation when training a machine learning model. Cross validation can be used to obtain a reliable estimate of machine learning model performance using only the training data set 720, and without using the validation data set 725, such as by splitting the training data set 720 into a number of groups (e.g., based on operator input that identifies the number of groups and/or based on randomly selecting a number of groups) and using those groups to estimate model performance. For example, using k-fold cross-validation, observations in the training data set 720 may be split into k groups (e.g., in order or at random). For a training procedure, one group may be marked as a hold-out group, and the remaining groups may be marked as training groups. For the training procedure, the machine learning system may train a machine learning model on the training groups and then test the machine learning model on the hold-out group to generate a cross-validation score. The machine learning system may repeat this training procedure using different hold-out groups and different test groups to generate a cross-validation score for each training procedure. In some implementations, the machine learning system may independently train the machine learning model k times, with each individual group being used as a hold-out group once and being used as a training group k−1 times. The machine learning system may combine the cross-validation scores for each training procedure to generate an overall cross-validation score for the machine learning model. The overall cross-validation score may include, for example, an average cross-validation score (e.g., across all training procedures), a standard deviation across cross-validation scores, or a standard error across cross-validation scores.

In some embodiments, the machine learning system may perform cross-validation when training a machine learning model by splitting the training set into a number of groups (e.g., based on operator input that identifies the number of groups and/or based on randomly selecting a number of groups). The machine learning system may perform multiple training procedures and may generate a cross-validation score for each training procedure. The machine learning system may generate an overall cross-validation score for each hyperparameter set 740 associated with a particular machine learning algorithm. The machine learning system may compare the overall cross-validation scores for different hyperparameter sets 740 associated with the particular machine learning algorithm and may select the hyperparameter set 740 with the best (e.g., highest accuracy, lowest error, or closest to a desired threshold) overall cross-validation score for training the machine learning model. The machine learning system may then train the machine learning model using the selected hyperparameter set 740, without cross-validation (e.g., using all of data in the training data set 720 without any hold-out groups), to generate a single machine learning model for a particular machine learning algorithm. The machine learning system may then test this machine learning model using the validation data set 725 to generate a performance score, such as a mean squared error (e.g., for regression), a mean absolute error (e.g., for regression), or an area under receiver operating characteristic curve (e.g., for classification). If the machine learning model performs adequately (e.g., with a performance score that satisfies a threshold), then the machine learning system may store that machine learning model as a trained machine learning model 745 to be used to analyze new observations, as described below in connection with FIG. 7.

In some implementations, the machine learning system may perform cross-validation, as described above, for multiple machine learning algorithms (e.g., independently), such as a regularized regression algorithm, different types of regularized regression algorithms, a decision tree algorithm, or different types of decision tree algorithms. Based on performing cross-validation for multiple machine learning algorithms, the machine learning system may generate multiple machine learning models, where each machine learning model has the best overall cross-validation score for a corresponding machine learning algorithm. The machine learning system may then train each machine learning model using the entire training data set 720 (e.g., without cross-validation), and may test each machine learning model using the validation data set 725 to generate a corresponding performance score for each machine learning model. The machine learning model may compare the performance scores for each machine learning model and may select the machine learning model with the best (e.g., highest accuracy, lowest error, or closest to a desired threshold) performance score as the trained machine learning model 745.

As indicated above, FIG. 7 is provided as an example. Other examples may differ from what is described in connection with FIG. 7. For example, the machine learning model may be trained using a different process than what is described in connection with FIG. 7. Additionally, or alternatively, the machine learning model may employ a different machine learning algorithm than what is described in connection with FIG. 7, such as a Bayesian estimation algorithm, a k-nearest neighbor algorithm, an a priori algorithm, a k-means algorithm, a support vector machine algorithm, a neural network algorithm (e.g., a convolutional neural network algorithm), and/or a deep learning algorithm.

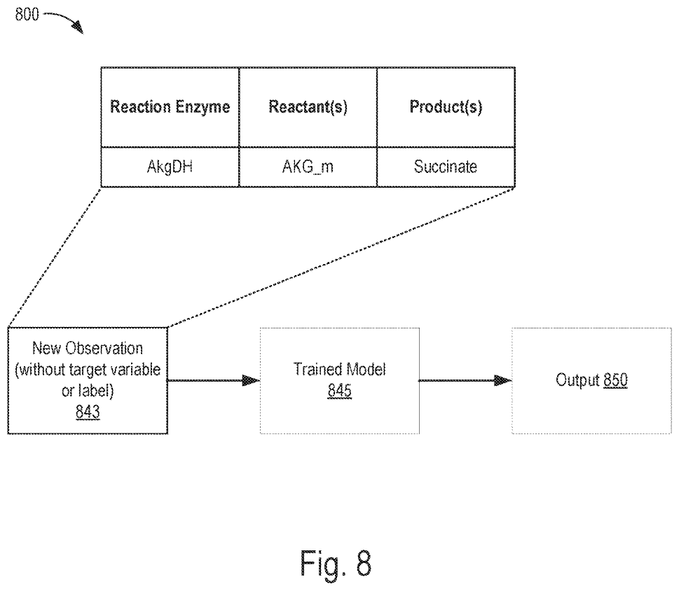

FIG. 8 is a diagram illustrating an example of applying a trained machine learning model to a new observation associated with characterizing a metabolic flux, such as the metabolic flux measuring system 610 of FIG. 6. The new observation (e.g., the result of the metabolic flux measuring system 610 determining flux rates for a metabolic pathway) may be input to a machine learning system that stores a trained machine learning model 845. In some embodiments, the trained machine learning model 845 may be the trained machine learning model 745 described above in connection with FIG. 7. The machine learning system may include or may be included in a computing device, a server, or a cloud computing environment, such as the metabolic flux analysis/profiling system 630 of FIG. 6.

As shown by reference number 810, the machine learning system may receive a new observation (or a set of new observations) and may input the new observation to the machine learning model 800. As shown, the new observation may include a first feature of an enzyme catalyzing a metabolic reaction, a second feature of enzyme reactant(s), a third feature of enzyme product(s), and so on. As shown, for the new observation, the first feature may have a value of “AkgDH,” the second feature may have a value of “Akg_m,” the third feature may have a value of “Succinate,” and so on. In some embodiments, the new observation may be without a target variable or label, and the trained machine learning model may be used to predict the target variable or label, e.g., predict a metabolic flux rate for a metabolite of interested as described herein.

The machine learning system may apply the trained machine learning model 845 to the new observation to generate an output 850 (e.g., a result). The type of output may depend on the type of machine learning model and/or the type of machine learning task being performed. For example, the output may include a predicted (e.g., estimated) value of target variable (e.g., a value within a continuous range of values, a discrete value, a label, a class, or a classification), such as when supervised learning is employed. Additionally, or alternatively, the output may include information that identifies a cluster to which the new observation belongs and/or information that indicates a degree of similarity between the new observation and one or more prior observations (e.g., which may have previously been new observations input to the machine learning model and/or observations used to train the machine learning model), such as when unsupervised learning is employed. In some implementations, the output 850 includes an indication of an identity of an item in the presently scanned container. For example, the output can correspond to a “best guess” for what item(s) the presently scanned container includes, based on the input features. Furthermore, as described herein, in some cases, the output 850 includes a confidence value for the output.

In some embodiments, the trained machine learning model 845 may predict a value of “Flux Rate #1” and “96%” for the target variable for the new observation, indicating that there is a 96% likelihood that the container includes the item “Flux Rate #1.” Based on this prediction (e.g., based on the value having a particular label or classification or based on the value satisfying or failing to satisfy a threshold), the machine learning system may provide a recommendation and/or output for determination of a recommendation, such a recommendation regarding growth rate of a cell or cells in a particular growth media, a recommendation to store the output in storage (e.g., the metabolic flux measuring system 610), among other examples. Additionally, or alternatively, the machine learning system may perform an automated action and/or may cause an automated action to be performed (e.g., by instructing another device to perform the automated action), such as indicating that a cell or cells are growing at a satisfactory rate (when a confidence satisfies a confidence threshold) or outputting an instruction to supplement the growth media (when a confidence does not satisfy a confidence threshold).

As another example, if the machine learning system were to predict a value of “LOW” for the target variable of “confidence value,” then the machine learning system may provide a different recommendation and/or may perform or cause performance of a different automated action (e.g., output an indication to manually review). In some implementations, the recommendation and/or the automated action may be based on the target variable value having a particular label (e.g., classification or categorization) and/or may be based on whether the target variable value satisfies one or more threshold (e.g., whether the target variable value is greater than a threshold, is less than a threshold, is equal to a threshold, or falls within a range of threshold values).

In this way, the machine learning system may apply a rigorous and automated process to, for example, optimize growth conditions for a cell or cells accurately and efficiently through metabolic flux analysis. The machine learning system enables recognition and/or identification of tens, hundreds, thousands, or millions of features and/or feature values for tens, hundreds, thousands, or millions of observations, thereby increasing accuracy and consistency and reducing delay associated with the relative resources (e.g., computing or human) required to be allocated for tens, hundreds, or thousands of operations necessary to manually analyze metabolic flux. As indicated above, FIG. 8 is provided as an example. Other examples may differ from what is described in connection with FIG. 8.

In accordance with the present disclosure, the machine learning model 700 serves as an illustrative example of a computational model capable of executing the desired functions, but it should be understood that other computational models can be employed either as supplementary to or as replacements for the machine learning model 700. For example, other computational models can include, but are not limited to, as decision trees, support vector machines, Gaussian processes, genetic algorithms, GPT (Generative Pre-trained Transformer), or Artificial Neural Networks (ANNs). In some cases, a computational model can be configured to employ bounded range calculations for determining the flux rate between values X and Y for a metabolite of interest, solving a function that dynamically adjusts the flux rate based on provided input data. The framework herein described is compatible with any of these or other computational models. Thus, the mention of NNs or any other specific computational model is neither intended to circumscribe the existing functionalities nor to inhibit future advancements involving diverse computational methodologies.

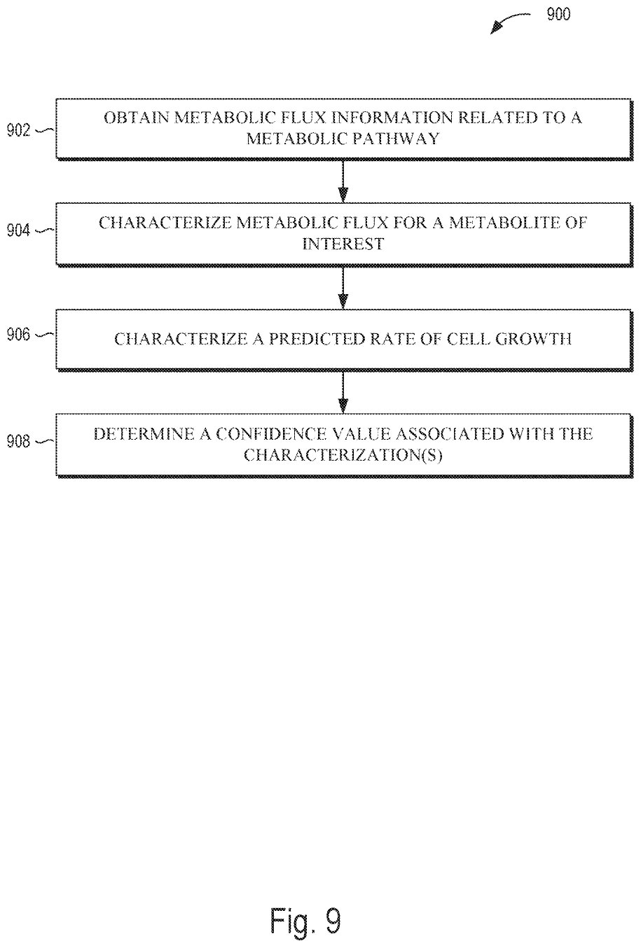

FIG. 9 is a flow diagram illustrative of an embodiment of a process 900 for application of the machine learning model described herein to, e.g., optimizing growth conditions for a cell or cells using metabolic flux analysis. Although described as being implemented by the metabolic flux analysis/profiling system 630, it will be understood that the elements outlined for process 900 can be implemented by one or more computing devices or components that are associated with the metabolic flux analysis system 600, such as, but not limited to, the metabolic flux measuring system 610 or the metabolic flux analysis/profiling system 630. Thus, the following illustrative embodiment should not be construed as limiting.

At block 902, the metabolic flux analysis/profiling system 630 receives or otherwise obtains metabolic flux information related to a metabolic pathway. The metabolic pathway can include one or more chemical reactions, such as one or more enzymatically catalyzed chemical reactions. As described herein, the metabolic flux analysis system 600 can analyze metabolic flux of one or more products and reactants.

At block 904, the metabolic flux analysis/profiling system 630 receives or otherwise characterizes metabolic flux for a metabolite of interest. As described herein, in some cases, at least a portion of the data is measured and stored locally, such as in a local data store. In addition, or alternatively, at least a portion of the data may be stored remotely, such as in a means for remote data storage 612. As such, depending on the location of the flux data, the metabolic flux analysis/profiling system 630 obtains the metabolic flux data from the local storage and/or from the remote storage. As described herein, the metabolic flux data may include metabolite flux rates of one or more reactants of products along a metabolic pathway, reactant consumption rates, product excretion rates resulting from cell growth, etc. The measured/obtained metabolic flux rates may then be characterized to determine a predicted value for the flux rate of a metabolite of interest.

At block 906, the metabolic flux analysis/profiling system 630 may then utilize the predicted metabolic flux rate(s) of a metabolite(s) of interest to characterize a predicted rate of cell growth, for example, a rate of cell growth in a particular cell media. In some cases, the metabolic flux analysis/profiling system 630 utilizes a machine learned algorithm to characterize and predict the metabolic flux rate(s) of the metabolite(s) of interest, and/or to predict the cellular growth rate of the cell or cells under particular growth conditions.

At block 908, the container management system 130 determines a confidence value associated with the characterization at block 606. In some cases, the confidence value corresponds to the degree to which the predicted metabolic flux rate matches flux rates associated/observed under particular growth conditions. For example, if the metabolic flux analysis/profiling system 630 returns a indicates that the predicted metabolic flux rate for a metabolite of interest matches the associated/observed metabolic flux rate for the metabolite of interest for the cell or cells growing under optimal conditions, then the metabolic profiling system may return a relatively high confidence score that the cell or cells are being grown under optimal conditions, and modifying/supplementing the media may not be necessary. In another example, if the predicted metabolic flux rate for the metabolite of interest does not match the associated/observed metabolic flux for the metabolite of interest for the cell or cells growing under optimal conditions, then the metabolic flux analysis/profiling system 630 may return a relatively low confidence score that the cells are being grown under optimal conditions, and/or the cell media may need to be modifies/supplemented to achieve optimal growth conditions. In some embodiments, the metabolic flux analysis/profiling system 630 utilizes a machine learned algorithm to determine a confidence value.

In some cases, the metabolic flux analysis/profiling system 630 determines a confidence value in accordance with a confidence generation policy. As part of generating the confidence value, the metabolic flux analysis/profiling system 630 can obtain a confidence generation policy, which can be stored locally or remotely, e.g., on a network 620.

The confidence generation policy can indicate how to generate a confidence value. For example, the confidence generation policy can include a particular set of rules or instructions relating to assigning weighting values to measured metabolic flux rates. For example, the confidence generation policy can indicate that the measured flux rate of a first metabolite should be weighted more heavily in a confidence value determination than the measured flux rate of a second metabolite in predicting the flux rate of the metabolite of interest.

At block 910, the metabolic flux analysis/profiling system 630 causes an action based on the confidence value. In some cases, if the confidence value indicates a satisfactory level of confidence, then action includes accepting that the cell or cells are being grown under optimal conditions. For example, the action can include storing an indication that the cell growth media does not require further supplements for optimal cell growth. As another example, if the confidence value indicates an unsatisfactory level of confidence, then action includes a suggestion that further optimization of the cell growth media, and/or adding supplements to the cell growth media, may be necessary to achieve optimal cell growth.

It will be understood that the various blocks described with respect to FIG. 9 can be implemented in a variety of orders and/or can be implemented concurrently or in an altered order, as desired. For example, in some cases, the process 900 can be concurrently performed on multiple cultures of cell or cells, such as tens, hundreds, or thousands of cultures. In some cases, one or more blocks occur in real-time or near-real time. Furthermore, it will be understood that fewer, more, or different blocks can be used as part of the data flow of FIG. 9.

In summary, using spent media data along with cell growth and the concepts of metabolic flux analysis provide a more in-depth picture of how specific cells consume and utilize raw materials to support cell growth, e.g., as shown in FIG. 4. These data can in turn be used to develop and/or optimize media for cell growth as well as be used to determine a feeding regime for cells in a bioreactor for larger scale cell growth. These data can also be used to define the solution space for digital twin models to predict flux rates. These predicted flux rates can then be used to further enhance media, suggest genetic alterations to improve desired traits and/or to accelerate selection of cell strains or clones with improved propensity for desired traits. These predicted fluxes can also be used to diagnose diseases and inform drug treatment strategies.

Furthermore, methods and systems described herein can be used to calculate metabolic flux rates through spent cell media analysis and/or cell extracts analysis to direct cell-specific media optimization, control of fermentation systems or other cell culture systems and/or direct a cell-specific feeding regime for cultured cells as described herein. These methods and systems can further be used to provide a cell culture medium, a cell culture medium supplement, and/or a cell-specific feeding regime, without departing from the scope of the inventive concept.

### EXAMPLE EMBODIMENTS

Various examples of methods, systems, or digital twin platforms for guiding cell-specific media determinations, metabolic flux analysis, or control of various cell culture systems can be found in the following clauses:
Clause 1. A method of utilizing spent cell media analysis and/or cell extracts analysis for metabolic flux analysis (MFA) to direct cell-specific media optimization, control of fermentation systems or other cell culture systems and/or a cell-specific feeding regime for cultured cells as described herein.
Clause 2. The method of Clause 1, wherein the method further comprises: subjecting spent cell media analysis and/or cell extracts analytical data to a set of algorithms and regressions to calculate a limited number of flux rates, with or without the use of a stable isotope tracer; using a combination of data set generation and computer-assisted processing to identify various cell media component limitations and/or possible excesses; and determining components for optimizing culture medium for a specific cell type or providing a feeding regime for a specific cell type.
Clause 3. The method of Clause 2, wherein the specific cell type is a prokaryotic or eukaryotic cell.
Clause 4. The method of Clause 3, wherein the cell is a plant cell, animal cell to include both human and non-human, bacterium, fungus or mold, or transformant, transfectant or cell line derived therefrom.
Clause 5. The method of Clause 4, wherein the cell is differentiated, partially differentiated or undifferentiated.
Clause 6. The method of any one of Clauses 1-5, wherein the method comprises utilizing spent cell media and cell extracts.
Clause 7. The method of any one of Clauses 1-5, wherein the method comprises utilizing spent cell media only.
Clause 8. The method of any one of Clauses 1-5, wherein the method comprises utilizing cell extracts only.
Clause 9. The method of any one of Clauses 1-8, wherein the method does not comprise use of a stable isotope tracer.
Clause 10. The method of any one of Clauses 1-8, wherein the method comprises use of a stable isotope tracer.
Clause 11. The method of Clause 10, wherein the stable isotope is 13C 15N, 2H or 18O.
Clause 12. A cell culture medium produced by the method of any one of Clauses 1-11.
Clause 13. A cell culture medium supplement produced by the method of any one of Clauses 1-11.
Clause 14. A cell-specific feeding regime developed by using the method of any one of Clauses 1-11.
Clause 15. A system comprising a computer program to calculate metabolic flux rates utilizing spent cell media analysis and/or cell extracts analysis to direct cell-specific media optimization, control of fermentation systems or other cell culture systems and/or direct a cell-specific feeding regime for cultured cells as described herein.
Clause 16. A cell culture system, wherein the culture is, for example, but not limited to, a batch culture, a fed-batch culture, a perfusion culture, etc.
Clause 17. A metabolic model created by a method of calculating flux rates with or without the use of stable isotopes.
Clause 18. MFA rates can be used to define the solution space for digital twins using the metabolic model defined in Clause 17.
Clause 19. The MFA rates of Clause 18 may be acquired from quantitative analytical techniques including mass spectrometry, NMR, Ramen spectroscopy, or bioanalytical methods including ELISA, Northern, Southern, Western Blot, electrochemical, fluorescence, luminescence, or colorimetric
Clause 20. A method of predicting cell strain, cell clone selection, informing appropriate genetic modifications, or diagnosing and/or treating diseases including using predicted flux rates derived from a digital twin is described herein.
Clause 21. A method and platform utilizing the concepts of metabolic flux rate calculations and analysis (MFA) within a digital twin to direct cell-specific media optimization, control of fermentation systems or other cell culture systems, a cell-specific feeding regime for cultured cells, predict cell strain or cell clone selection, inform appropriate genetic modifications, maximize bioproduction output, and/or diagnose, inform, and treat diseases and disease states as described herein.
Clause 22. A digital twin system for predicting metabolic flux rates in a culture system, comprising: a metabolic model; a defined metabolic solution space; a means for inputting measured experimental data into the digital twin system; and a means for generating predictive flux rates within the defined solution space based on the input experimental data and the metabolic model.
Clause 23. The digital twin system of Clause 22, wherein the predictive flux rates are constrained by the experimentally measured inputs and the defined solution space.
Clause 24. The digital twin system of Clause 22 or 23, further comprising means for performing side-by-side analysis of a culture system, either during an active cell culture or as a post-culture analysis and generating culture-specific output.
Clause 25. The digital twin system of Clause 22-24, wherein the culture-specific output comprises insights applicable to an active culture system or future cultures sharing features with the culture analyzed by the digital twin.
Clause 26. A metabolic model for use in a digital twin system, comprising a mapped connection of enzymatic pathways, wherein the flow of nutrients within the mapped system can be tracked from input to output and final products including ‘biomass.’
Clause 27. A metabolic model of Clause 26 for the use in the digital twin must contain at least two (2) nodes where at least one (1) node is constrained by measured experimental data
Clause 28. The metabolic model of Clause 26, wherein the enzymatic pathways can map entire enzymatic chains or simplified enzymatic chains following known reaction paths defined in current literature.
Clause 29. The metabolic model of Clause 26-29, further encompassing the use of enzyme kinetics and enzymatic properties to calculate substrate-product concentrations.
Clause 30. A defined solution space for the digital twin system in Clause 22 comprises the range of possible metabolic flux rates. The solution space can arise from any individual point or combinations of the following points, wherein said flux rate solution space represents metabolic flux rates of cells within a specific context: quantified metabolite data derived from intracellular and/or extracellular sources for a specific experimental design; a computationally trained metabolic model; a repository of flux data; inherent enzymatic properties, including but not limited to kinetics, regulation, binding affinity; and combinations of genetic mapping with -omics data.
Clause 31. The method of Clause 21, further comprising supplementing the defined flux rate solution space with experimental data obtained from a specific cell culture process used to further constrain the solution space, wherein the number of experimental data points is greater than or equal to two (2) to further constrain the method in Clause 21.
Clause 32. The digital twin system may include a means for training and validating the metabolic model using data sets to refine the metabolic model's solution space and generate flux outputs.
Clause 33. The digital twin system of Clause 32, wherein the training and validating data sets are used to confirm the successful training of the model with validation data sets.
Clause 34. The method of the inventive concept may further include the use of cellular culture data from collection of spent media, cell extract data, and/or cell growth data.
Clause 35. The digital twin system of the inventive concept, wherein the predetermined output of the model and solution space consists of calculated flux rates.
Clause 36. The digital twin system of the inventive concept, wherein the digital twin output encompasses a range of outputs including reporting flux rates, interpreting and graphically analyzing flux rates, or executing external factors to make corrections to an active culture system.
Clause 37. The digital twin fluxes of the inventive concept may utilize both the defined solution space and experimentally defined measurements to calculate metabolic flux rates.
Clause 38. The method of the inventive concept, wherein the method further comprises: subjecting spent cell media analysis and/or cell extracts analytical data to a set of algorithms and regressions to calculate a limited number of flux rates, with or without the use of a stable isotope tracer.
Clause 39. The digital twin method of the inventive concept, further comprising the utilization of the calculated metabolic flux rates to optimize cell-specific media composition in a cell culture process.
Clause 40. The digital twin method of the inventive concept, further comprising the utilization of the calculated metabolic flux rates to control fermentation systems in a cell culture process.
Clause 41. The digital twin method of the inventive concept, further comprising the utilization of the calculated metabolic flux rates to direct a cell-specific feeding regime for cultured cells, including the use of computer-assisted methods to calculate metabolic flux rates.
Clause 42. A system for optimizing cell culture processes comprising of defining a flux rate solution space based on experimental data, data used to train a computational model, and a repository of flux data, wherein said flux rate solution space represents metabolic flux rates of cells within a specific context.
Clause 43. The digital twin method of the inventive concept, further comprising means for supplementing the defined flux rate solution space with experimental data obtained from a specific cell culture process.
Clause 44. The digital twin method of the inventive concept, further comprising the means for utilizing the calculated flux rates to optimize cell-specific media composition in a cell culture process.
Clause 45. The digital twin method of the inventive concept, further comprising the means for utilizing the calculated flux rates to control fermentation system or cell culture reactors in a cell culture processes.
Clause 46. The system of Clause 15, further comprising: using a combination of data set generation and computer-assisted processing to identify various cell media component limitations and/or excesses; and determining components for optimizing culture medium for a specific cell type or providing a feeding regime for a specific cell type.

### Terminology

Although this disclosure has been described in the context of some cases and examples, it will be understood by those skilled in the art that the disclosure extends beyond the disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the embodiments described above. Accordingly, unless otherwise stated, or unless clearly incompatible, each embodiment of this inventive concept may include, additional to its essential features described herein, one or more features as described herein from each other embodiment of the inventive concept disclosed herein.

Features, materials, characteristics, or groups described in conjunction with an aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, some features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in some combinations, one or more features from a claimed combination can, in some cases, be excised from the combination, and the combination may be claimed as a subcombination or variation of a sub combination.

Moreover, while operations may be depicted in the drawings or described in the specification in an order, such operations need not be performed in the order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some cases, the actual steps taken in the processes illustrated and/or disclosed may differ from those shown in the figures. Depending on the embodiment, some of the steps described above may be removed, others may be added. Furthermore, the features and attributes of the embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, some aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

The term “comprise,” as used herein, in addition to its regular meaning, may also include, and, in some embodiments, may specifically refer to the expressions “consist of” and/or “consist essentially of.” Thus, the expression “comprise” can also refer to, in some embodiments, the specifically listed elements of that which is claimed and does not include further elements (“consist of”), or embodiments in which the specifically listed elements of that which is claimed may encompass further elements that do not materially affect the basic and novel characteristic(s) of that which is claimed (“consist essentially of”), as well as embodiments in which the specifically listed elements of that which is claimed may and/or does encompass further elements.

Conditional language, such as “can,” “could,” “might,” or “may,” unless specifically stated otherwise, or otherwise understood within the context as used, is generally intended to convey that some cases include, while other embodiments do not include, some features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that some cases require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount. As another example, in some cases, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

The foregoing is illustrative of the present invention and is not to be construed as limiting thereof.

1. A method for predicting flux rate of a metabolite of interest in a cell comprising:
obtaining quantitative measurements or analytical values for components in spent media or cell extracts;
inputting an indication of the quantitative measurements or the analytical values into a trained neural network, wherein the trained neural network characterizes metabolic flux in the cell based on the quantitative measurements or the analytical values; and
obtaining a predicted flux rate for the metabolite of interest as an output from the trained neural network,
wherein the output is utilized to characterize how the cell behaves or grows under a first condition.

2. The method of claim 1, wherein the components in spent media or cell extracts comprise amino acids, fatty acids, sugars, vitamins, minerals, organic acids, or growth factors.

3. The method of claim 1, further comprising:
obtaining temporal cellular growth metrics indicative of a rate of cellular metabolism, cellular expansion, or proliferation of the cell over a defined time period; and
inputting an indication of the temporal cellular growth metrics into the trained neural network, wherein the characterization of the metabolic flux by the trained neural network is further based on the temporal cellular growth metrics.

4. The method of claim 1, further comprising:
obtaining compositional data corresponding to a biomass composition of the cell; and inputting an indication of the compositional data into the trained neural network,
wherein the characterization of the metabolic flux by the trained neural network is further based on the compositional data.

5. The method of claim 1, wherein the quantitative measurements or analytical values comprises measurements of components in spent media or cell extracts, cell growth, rate of production of cell products, or biomass composition.

6. The method of claim 1, further comprising normalizing the quantitative measurements or the analytical values against a control to create normalized values, wherein the indication of the quantitative measurements or the analytical values comprises the normalized values.

7. The method of claim 1, wherein the cell is selected from the group consisting of prokaryotic cells, eukaryotic cells, plant cells, animal cells, bacterial cells, fungi, and molds.

8. The method of claim 1, wherein the cell includes a human cell.

9. The method of claim 1, wherein the cell includes a non-human cell.

10. The method of claim 1, wherein the metabolite of interest is selected from the group consisting of amino acids, fatty acids, and sugars.

11. The method of claim 1, wherein the quantitative measurements or the analytical values are determined using a stable isotope tracer.

12. The method of claim 10, wherein the stable isotope tracer is 13C, 15N, 2H or 180.

13. The method of claim 1, wherein the quantitative measurements or the analytical values are determined in only spent cell media.

14. The method of claim 1, wherein the quantitative measurements or the analytical values are determined in only cell extracts.

15. The method of claim 1, wherein the predicted flux rate for the metabolite of interest is used to determine how the cell will behave or grow in a particular cell media.

16. The method of claim 1, wherein the predicted flux rate for the metabolite of interest in the cell is used to select for a cell that grow more rapidly or efficiently in a particular media.

17. The method of claim 1, wherein the predicted flux rate for the metabolite of interest in the cell is used to optimize a cell media for the cell.

18. The method of claim 16, wherein the cell media is optimized by adding a cell media supplement.

19. The method of claim 17, wherein the cell media supplement is selected from the group consisting of amino acids, vitamins, organic acids, lipids, carbohydrates, hormones, growth factors, cytokines, attachment factors, antibiotics, plant extracts, hydrolysates yeast extracts, hydrolysates, and serum.

20. The method of claim 1, wherein the predicted flux rate comprises a predicted consumption, degradation, or excretion of the metabolite of interest.

21. The method of claim 1, wherein the trained neural computational model has been pre-conditioned utilizing a dataset comprising representative metrics pertinent to cellular metabolic pathways, rates of cellular expansion, proliferative tendencies, or growth kinetics.

22. A cell culture medium selected by a method comprising the method of any one of claims 1-21.

23. A cell culture medium supplement selected by a method comprising the method of any one of claims 1-21.

24. A cell-specific feeding regime selected by a method comprising the method of any one of claims 1-21.

25. A system for predicting flux of a metabolite of interest in a cell comprising:
at least one processor;
a circuit coupled to the at least one processor and configured to input quantitative measurements or analytical values for components in spent media or cell extracts;
a circuit configured to characterize the quantitative measurements or analytical values for components in spent media or cell extracts with a trained neural network;
an input/output (I/O) circuit coupled to the at least one processor;
a storage circuit coupled to the at least one processor and configured to store data and parameters; and
a memory coupled to the at least one processor comprising computer-readable program code stored in the memory that when executed by the at least one processor causes the at least one processor to perform operations comprising:
characterizing quantitative measurements or analytical values for components in spent media or cell extracts;
obtaining a predicted flux rate for a metabolite of interest from characterization of the quantitative measurements or analytical values for components in spent media or cell extracts with the trained neural network; and
controlling output of a determination of a predicted flux rate for the metabolite of interest by way of the I/O circuit,
wherein the output is utilized to characterize how the cell behaves or grows under a first condition.

26. A computer-implemented method for predicting flux of a metabolite of interest in a cell comprising:
inputting quantitative measurements or analytical values for components in spent media or cell extracts into a trained neural network;
retrieving representative data related to cell metabolism, cellular expansion, cellular proliferation, and/or cellular growth metrics from a storage circuit into the trained neural network;
characterizing the quantitative measurements or analytical values for components in spent media or cell extracts with the trained neural network, wherein the trained neural network characterizes metabolic flux in the cell based on the quantitative measurements or the analytical values and the representative data related to cell metabolism, cellular expansion, cellular proliferation, and/or cellular growth metrics;
outputting a predicted flux rate for a metabolite of interest from characterization of the quantitative measurements or analytical values for components in spent media or cell extracts,
wherein the output is utilized to characterize how the cell behaves or grows under a first condition.
